(19) United States
(12) Patent Application Publication
(10) Pub. No.: US 2010/0130860 A1
(43) Pub. Date: May 27, 2010

(54) MEDICAL IMAGE-PROCESSING DEVICE, MEDICAL IMAGE-PROCESSING METHOD, MEDICAL IMAGE-PROCESSING SYSTEM, AND MEDICAL IMAGE-ACQUIRING DEVICE

(75) Inventor: Hitoshi YAMAGATA, Otawara-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-shi (JP)

(21) Appl. No.: 12/618,968

(22) Filed: Nov. 16, 2009

(30) Foreign Application Priority Data
Nov. 21, 2008 (JP) 2008-298599

(51) Int. Cl. A61B 8/14; G06K 9/00

(52) U.S. Cl. 600/443; 382/128

(57) ABSTRACT

A medical image-processing device stores the parameters of window level and window width set by a window conversion part. An opacity curve-setting part sets the settings of the OWL and OWW in an opacity curve based on the stored parameters during volume rendering for volume data. In this way, the medical image-processing device sets the opacity of a three-dimensional image.

### BACKGROUND OF THE INVENTION

1. Field of the Invention

The present invention relates to techniques regarding display condition settings for imaged medical image data.

2. Description of the Related Art

In medical institutions, after acquiring information of tissue within a subject—such as perspective images, tomographic images, and blood flow within the subject—using a medical image-acquiring device, the acquired tissue information is converted into medical images. Moreover, in medical institutions, examinations and diagnoses are conducted with such medical images. There are various types of this medical image-processing device. For example, there are X-ray CT (computed tomography) devices, MRI (magnetic resonance imaging) devices, ultrasound image-acquiring devices (ultrasound diagnostic equipment), nuclear medicine diagnostic devices (NM: nuclear medicine), PET-CT (positron emission tomography-computed tomography) devices, and others.

Moreover, these medical image-acquiring devices collect information of body tissue by capturing the subject. Furthermore, the medical image-acquiring device generates a medical image of the subject's body tissue from the information that has been collected.

For example, with an X-ray CT device, a scan is performed while rotating an X-ray tube and an X-ray detector. With this scan, the X-ray CT device detects X-rays that have penetrated the subject.

Moreover, the X-ray CT device performs a reconstruction process, etc. with the detected X-rays as projection data. Furthermore, the X-ray CT device generates a plurality of two-dimensional images or three-dimensional images by performing a reconstruction process.

Moreover, with an MRI device, by placing the subject in a static magnetic field, the nuclear spin (e.g., hydrogen atoms or proton) within the subject is oriented in the direction of the static magnetic field. Subsequently, the MRI device applies RF pulse (radio-frequency pulse) to the subject and excites its nuclear spin while applying a gradient magnetic field to provide positional information. The MRI device reconstructs an image with MR (Magnetic Resonance) signals generated from the subject in conjunction with this excitation and the spatial information thereof.

Moreover, with the ultrasound image-acquiring device, ultrasound waves are transmitted to a diagnostic region in the subject by an ultrasound probe. Subsequently, with the ultrasound image-acquiring device, reflected waves are received from tissue boundaries within the subject with different acoustic impedances. The ultrasound image-acquiring device scans the ultrasound waves with this ultrasound probe, obtains information of the subject's body tissue, and generates an image.

The medical image generated in this way will be a two-dimensional (2D) image showing a cross-section of the subject or a three-dimensional image (3D) image. With the X-ray CT device, helical scanning with an MDCT (multi-detector row CT) using multiple array detectors or a conventional scan with an ADCT (area detector CT) using more than 256-row detectors is performed. With these types of scanning by the X-ray CT device, data for generating three-dimensional images (hereafter referred to as “volume data”) is collected.

Moreover, with the MRI device, three-dimensional images have been generated based on a plurality of two-dimensional image data collected by multi slice imaging using the spin echo method.

Furthermore, with the MRI device, three-dimensional imaging has been performed by using phase encoding in the slice direction, in mainly using the fast gradient echo method (FGE). In addition, recently, the MRI device have been improved in the use of high magnetic fields, high performance of gradient magnetic field, increased number of channels of the transceiver coil array, high performance of parallel imaging, and other attempts. As a result, recently, the MRI device has reduced the time taken for three-dimensional imaging.

For the ultrasound image-acquiring device, a method of rotating or swaying an ultrasound transducer with one-dimensional array in an ultrasound probe has recently been used. This ultrasound transducer collects and displays ultrasound images in three dimensions. Moreover, for the ultrasound image-acquiring device, a system in which ultrasound images are collected and displayed in three-dimensions by an electrically scanning ultrasound probe of an ultrasound transducer with a two-dimensional (2D) array in which piezoelectric elements are arranged in a matrix state has been used. Such a medical image shown in three-dimensions is useful for the diagnosis of regions that are easy to miss in two-dimensional images, and improvements in diagnostic accuracy using the three dimensional medical images can be expected.

In addition, arbitrary cross-sectional images, such as MPR (multi planar reconstruction) images, will be required for observing arbitrary cross-sections such as those not represented in two-dimensional images.

Moreover, when displaying two-dimensional images and three-dimensional images generated by the medical image-processing device, image processing and adjustments of display conditions are performed by an operator of the device so that the images will be easy to view. For example, with the X-ray CT device, a pixel intensity based on a CT value (HU (Hounsfield unit)) is assigned to each pixel in an arbitrary cross-section. In two-dimensional images, according to each pixel intensity based on this CT value, a gray level (contrast), for example, is set.

In a two-dimensional image, a gray-scale according to the pixel intensity is set on the basis of each pixel. Here, in the case of the X-ray CT device, the CT value has been defined so that it will be 0 for water and −1,000 for air. However, the gray-scale in two-dimensional images may sometimes be represented with only a 256 gray-scale from 0 to 255, for example. In addition, in MR images and ultrasound images, a 256 gray-scale is insufficient to express all pixel intensities.

Therefore, conventionally, when setting display conditions for two-dimensional images, a window level (WL) and a window width (W W) have been set in order to identify images with low contrast. Once the window level and window width are set, when the medical image-processing device attempts to display a two-dimensional image, among the pixel intensities for each pixel included in the image data, the range of the pixel intensities to be displayed with a gray-scale is kept within a certain range. In this image data, the range of the pixel intensities to be displayed with a gray-scale is the window width. In addition, the pixel intensity corresponding to the median value of the gray-scale display is referred to as the window level.

Further, the medical image-processing device performs rendering on a three-dimensional image to project and display volume data on a two-dimensional plane, for example. In general, as this rendering processing, volume rendering (VR) is used because it contributes to observing the overall image, such as the conditions inside an object to be displayed.

In volume rendering with the medical image-processing device, volume data is constructed virtually on a three-dimensional space. This three-dimensional space has the coordinates (X, Y, Z). Here, information for each coordinate in the volume data is defined as voxel data. In addition, in volume rendering, an arbitrary viewpoint and the direction of a light ray with respect to the object to be displayed are defined while a projection plane for projecting three-dimensional volume data as a pseudo-three-dimensional image from the viewpoint, etc. is defined. Moreover, a line of vision from that viewpoint toward the projection plane is defined. Along with the definition of this line of vision, on the line of vision from the viewpoint toward the projection plane, a gray level on the projection plane is defined based on the voxel value of each voxel (pixel intensity at a voxel) in the volume data in the order of the voxels. In this way, in volume rendering with the medical image-processing device, a pixel intensity for each coordinate in the three-dimensional space is defined and the results thereof are projected on the projection plane. There are multiple lines of vision in volume rendering, and the medical image-processing device successively defines a pixel intensity for each line of vision in volume rendering in a similar manner to the described processing.

In volume rendering with the medical image-processing device, when defining the gray level on the projection plane, the opacity for each voxel value is set. In volume rendering, according to this opacity, the display state of the object from the viewpoint is defined. That is, settings are defined so that the defined light ray will penetrate or be reflected by the object when viewing the projection plane from the defined viewpoint. This causes a volume-rendering image (hereafter simply referred to as “three-dimensional image”) is expressed as a pseudo-three-dimensional image.

In a three-dimensional image, the opacity is set on the basis of each voxel value as described above. Conventionally, when setting display conditions for three-dimensional images, the OWL (opacity window level) and OWW (opacity window width) are set. In addition, the OWL and OWW are set to define an opacity curve. The OWW is a range of pixel intensities expressed with an opacity scale. In addition, the OWL is the median value of the range.

Conventionally, this opacity curve is set using a GUI (graphical user interface) or the like as shown in FIGS. 7-10 in Japanese published unexamined application No. H11-283052. On the GUI shown in this publication, the operator can set pixel intensities that will be the upper and lower limits of the OWW via a manipulation part.

Moreover, conventionally, techniques of creating and analyzing a histogram of the pixel intensities of a region of interest to set an opacity curve have been proposed (e.g., Japanese published unexamined application No. 2008-6274). Namely, the medical image-processing device described in this publication specifies a region of interest and performs statistical processing of the pixel intensities in an image of the specified region. Moreover, the medical image-processing device generates a histogram as a result of the statistical processing. When the histogram is generated, the medical image-processing device analyzes the histogram. Furthermore, the medical image-processing device sets an opacity curve based on the analysis results.

With these medical image-acquiring devices, an image viewer is required to operate setting of the opacity curve, independently from setting the display condition of the two-dimensional image. Thus, the image viewer must set a display condition of an image, requiring much time for these tasks. Because these tasks are inefficient among the image-viewing tasks, they may deteriorate the efficiency of radiogram interpretation or diagnostic imaging.

### SUMMARY OF THE INVENTION

The present invention has been devised in view of the situation described above. The object of the present invention is to provide a medical image-processing device, an ultrasonic image-acquiring device, and a medical image-processing method that can simplify the setting of an opacity curve for viewing tissue information of a subject acquired by the medical image-processing device as a three-dimensional image, thereby resolving the inefficiency of the setting task, and can furthermore improve the efficiency of diagnostic imaging.

The first aspect of the present invention is a medical image-processing device comprising: information acquiring part configured to acquire a window level value and a window width value of medical image data of a subject; opacity setting part configured to set a opacity curve of volume rendering based on the window level value and the window width value; and volume rendering part configured to apply volume rendering process to the medical image data based on the opacity curve set by the opacity setting part.

The second aspect of the present invention is an ultrasonic image-acquiring device comprising information acquiring part configured to acquire gain adjustment value and STC adjustment value when at least one of gain adjustment and STC adjustment of ultrasonic image collection of a subject is conducted; opacity setting part configured to set a opacity curve based on the gain adjustment value or the STC adjustment value; and volume rendering part configured to apply volume rendering process to the medical image data based on the opacity curve.

The third aspect of this present invention is a medical image-processing method comprising: acquiring a window level value and a window width value of medical image data of a subject; setting a opacity curve of volume rendering based on the window level value and the window width value; and applying volume rendering process to the medical image data based on the opacity curve.

According to the first through third aspects of the present invention, parameters utilized for gray-scale display of medical image data are utilized for parameters of opacity curve for volume rendering.

The inventor of the present invention has confirmed that the opacity curve set by the medical image-processing device according to the present invention is effective for setting the opacity in three-dimensional images. Namely, in the present invention, effective presetting of the opacity curve is done by performing gray-scale processing tasks such as window conversion, gain adjustment, and STC adjustment. Therefore, the display-adjustment tasks for the opacity of three-dimensional images by the image viewer can be minimized or omitted. Furthermore, it will be possible to resolve difficulties in the tasks for setting opacity during volume rendering. As a result, the burden placed on the image viewer can be reduced, and improving the operability of radiogram-interpreting operation allows the efficiency of radiogram interpretation and diagnostic imaging to be improved.

### BRIEF DESCRIPTION OF THE DRAWINGS

FIG. 1 is a schematic block diagram showing the schematic conformation of a medical image-processing device according to the first embodiment of the present invention.

FIG. 2(A) is an example of a graphs showing the window level and window width in window conversion.

FIG. 2(B) is a schematic diagram showing an example of the relationship between pixel intensity and opacity in volume rendering of a three-dimensional image.

FIG. 2(C) is a schematic diagram showing an example of the relationship between pixel intensity and opacity in volume rendering of a three-dimensional image.

FIG. 3 is a schematic diagram showing an overview of a window conversion-setting screen in the medical image-processing device according to the present embodiment.

FIG. 4 is a schematic diagram showing an example of the viewpoint, line of vision, and projection plane in volume rendering.

FIG. 5 is a flow chart representing a series of operations of the medical image-processing device for explaining the tasks through which a user, such as an operator, performs display processing of a three-dimensional image using the medical image-processing device according to the first embodiment.

FIG. 6 is a block diagram showing the schematic conformation of a medical image-processing system according to the second embodiment of the present invention.

FIG. 7 is a flow chart representing a series of operations of the medical image-processing device for explaining the tasks through which a user, such as an operator, performs display processing of a three-dimensional image using the medical image-processing device according to the second embodiment.

FIG. 8 is a block diagram showing the schematic conformation of a medical image-processing system according to the fourth embodiment of the present invention.

### DETAILED DESCRIPTION OF THE EMBODIMENTS

### First Embodiment

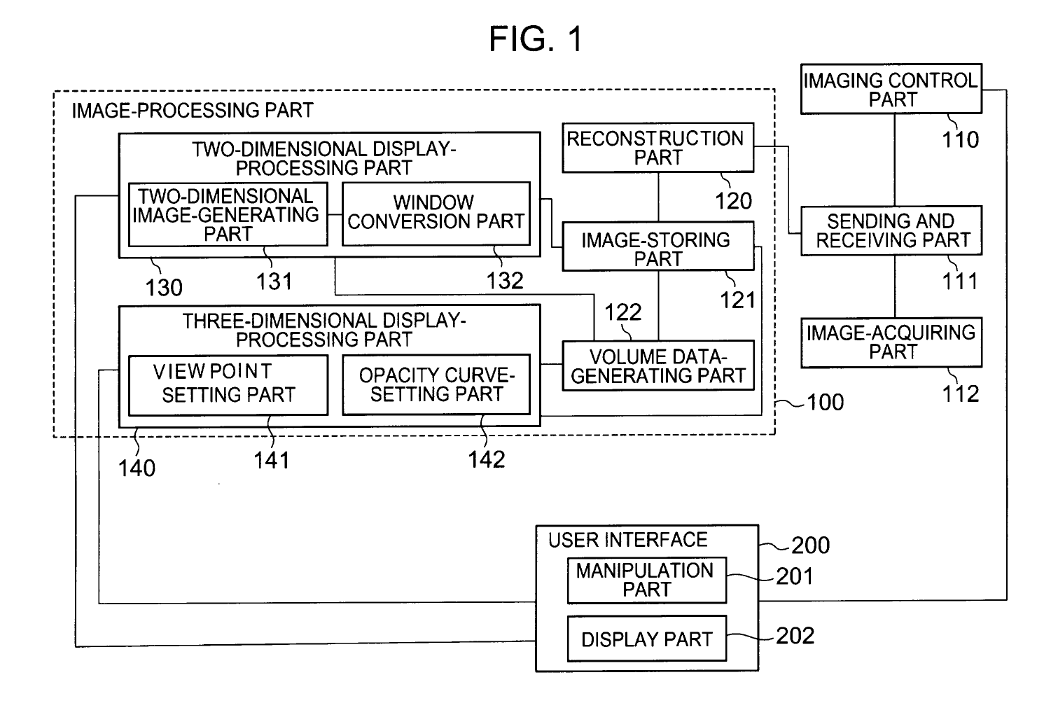

The medical image-processing device according to the first embodiment of the present invention is described as follows with reference to FIGS. 1-5. FIG. 1 is a schematic block diagram showing the schematic conformation of the medical image-processing device according to the first embodiment of the present invention. The first embodiment performs both two-dimensional display processing and three-dimensional display processing using the medical image-processing device.

In addition, the medical image-processing device according to the present embodiment performs not only display processing of image data but also imaging of a subject, reconstruction processing, and volume data generation. On the other hand, the medical image-processing device according to the present invention is not necessarily limited to a device performing these processes. The medical image-processing device according to the present invention may be one that performs only display processing, such as image processing of volume data that has been generated in advance, for example as in the fourth embodiment described below. Moreover, the medical image-processing device according to the present embodiment performs imaging of the subject, reconstruction processing, and volume data generation as well as two-dimensional image processing and three-dimensional image processing and is an example of the “medical image-processing device” according to the present invention.

### [Process of Volume Data Generation]

As shown in FIG. 1, an imaging control part 110 in the medical image-processing device of the first embodiment performs control related to imaging of the subject performed by an image-acquiring part 112 via a sending and receiving part 111. Namely, when the imaging control part 110 receives the settings for imaging conditions and an instruction to start imaging from the operator via a manipulation part 201, the imaging control part 110 sends a control signal related to imaging to the image-acquiring part 112 via the sending and receiving part 111. The image-acquiring part 112 receives the control signal and starts the process to image the subject under the imaging conditions that have been set based on the control signal.

Moreover, the medical image-processing device acquires tissue information indicating the conditions of tissues within the subject that has been acquired by imaging. This tissue information is an MR signal generated from the subject if the medical image-processing device is a MRI device, for example, and is an echo signal based on waves of ultrasonic pulse reflected from the subject if it is an ultrasound image-acquiring device. This tissue information is sent to the sending and receiving part 111 as an image signal. The sending and receiving part 111 performs processing on this image signal as appropriate and sends it to an image-processing part 100. When the image-processing part 100 receives the image signal from the sending and receiving part 111, this image signal is reconstructed as two-dimensional image data by a reconstruction part 120 in the image-processing part 100. The two-dimensional image data is stack data, for example.

The two-dimensional image data reconstructed in this way is stored in an image-storing part 121. This image-storing part 121 is composed of a hard disk, a memory, etc. This series of processes from imaging to image reconstruction is to be executed over time and the generated image data is successively stored in the image-storing part 121 in chronological order. In addition, in FIG. 1, the imaging control part 110 and the image-processing part 100 are displayed as separate configurations for convenience, but this is only one example. Namely, it does not prevent configuring these parts as one control part in the medical image-processing device.

A volume data-generating part 122 reads out the two-dimensional image data of a different position in the subject stored in the image-storing part 121 and generates volume data (voxel data group) represented in a three-dimensional real space. In addition, the volume data-generating part 122 may or may not perform interpolation processing of the two-dimensional image data when generating volume data. Moreover, if image data is collected using a medical image-processing device capable of collecting the volume data directly, the reconstruction part 120 generates volume data by performing reconstruction processing based on the image signal received from the sending and receiving part 111. Moreover, in this case, the volume data reconstructed by the reconstruction part 120 is stored in the image-storing part 121 and the volume data-generating part 122 does not perform the processing described above. In addition, the volume data-generating part 122, the imaging control part 110, and the image-acquiring part 112 are examples of the “imaging part” in the “medical image-acquiring device” according to the present invention.

### [Process of Two-Dimensional Display Processing]

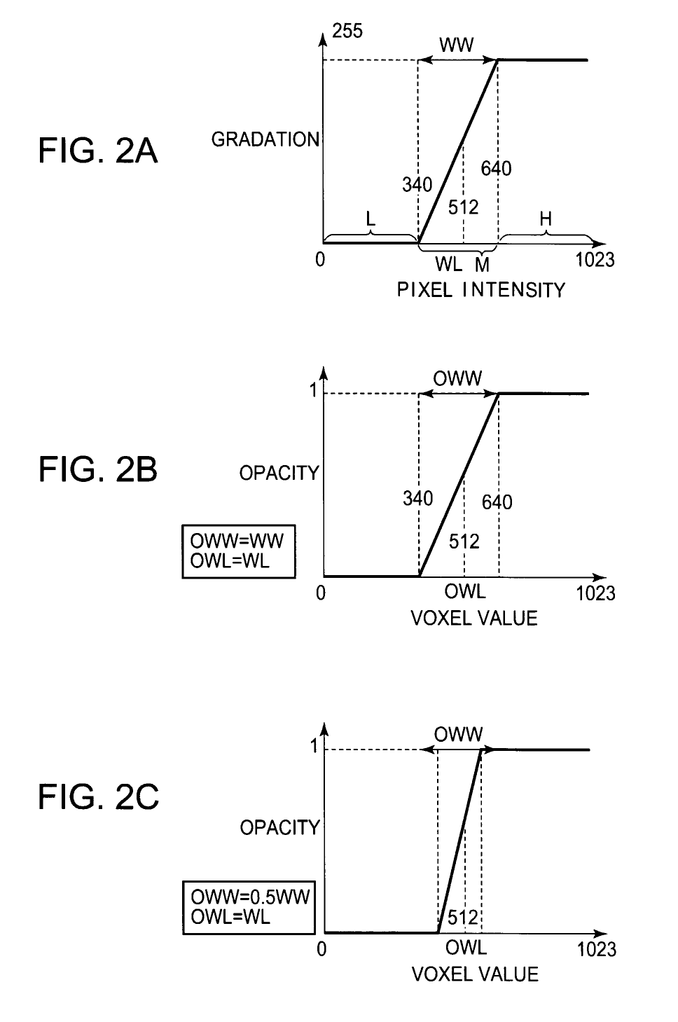

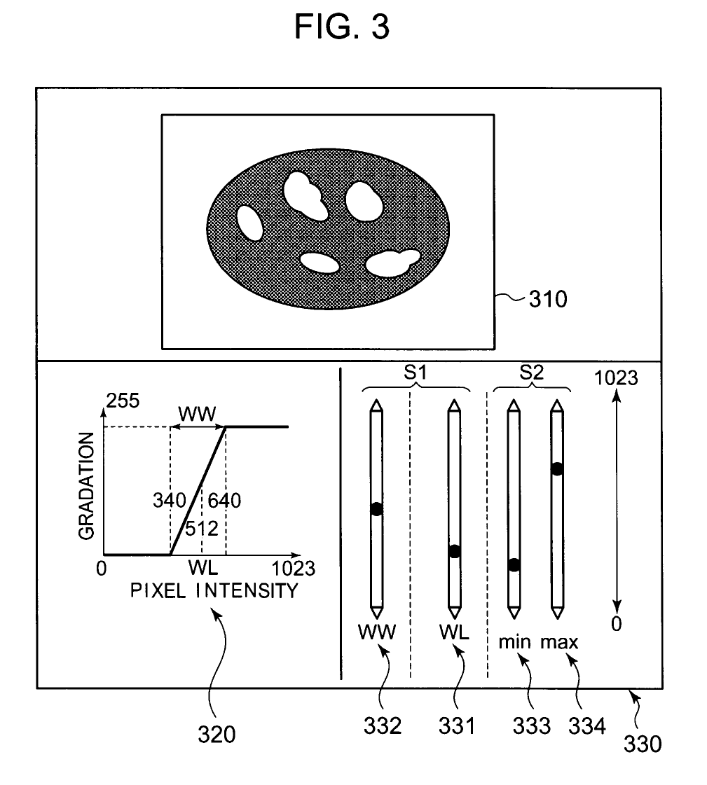

Next, image processing and the setting of display conditions (window conversion) related to a two-dimensional display are described using FIGS. 2(A) and 3. FIG. 2(A) is a schematic diagram showing an example of the relationship between the pixel intensity of the image signal and the gray-scale during window conversion of a two-dimensional image. FIG. 3 is a schematic diagram showing an example of a window conversion-setting screen in an embodiment according to the present embodiment. Herein, the pixel intensity is a value indicating the state of tissues in the subject, possessed by each voxel of the generated volume data.

A user interface 200 has a manipulation part 201 and a display part 202. Furthermore, the user interface 200 is configured including a display control part (not shown). The display control part transmits instruction information from the manipulation part 201 to the respective parts while transmitting the processing results of the respective parts to the display part 202.

As shown in FIG. 1, a two-dimensional display-processing part 130 is configured including a two-dimensional image-generating part 131 and a window conversion part 132. Among these, the two-dimensional image-generating part 131 performs two-dimensional image processing of volume data stored in the image-storing part 121 in response to manipulations by the operator via the manipulation part 201. As an example, a process of generating an MPR image by the two-dimensional image-generating part 131 will now be described. An MRP image is a useful image when the operator requires such an image in an arbitrary position and direction as a cross-section of the volume data.

When the operator attempts to view the MPR image, the operator performs manipulations for performing MPR processing via the manipulation part 201 in a processing selection screen (not shown) for two-dimensional image generation. Here, if the operator designates an arbitrary cross-section and performs a manipulation to cause three orthogonal cross-sections including this designated cross-section to be displayed, for example, the two-dimensional image-generating part 131 performs processing to display the three orthogonal cross-sections in response to the manipulation. Namely, the two-dimensional image-generating part 131 performs cross-section conversion processing of the volume data and generates and displays an axial image, a sagittal image, a coronal image, and an oblique image based on the designated cross-section.

The window conversion part 132 sets a window width (WW) and a window level (WL) in two-dimensional image data. This two-dimensional image data is data stored in the image-storing part 121 or data reconstructed and generated by the two-dimensional image-generating part 131. The window width is the width of a pixel intensity that should be displayed in gray-scale as a two-dimensional image among the respective pixel intensities assigned to each pixel in the two-dimensional image data. The window level is the pixel intensity that is centered in the window width. In addition, for gray-scale display of medical images with the medical image-processing device of the present embodiment, cases where it is possible to adjust the density of black and white components in an image and the 256-stage gray-scale to which values of 0 to 255 have been assigned are exemplified. In addition, the gray-scale mentioned herein may also be referred to a display brightness value of the image.

Further, the window conversion part 132 may conduct window conversion of tomographic images.

The window conversion performed by the operator using the medical image-processing device of the present embodiment will now be described with reference to FIGS. 2(A) and 3. FIG. 2(A) is an example of a graph showing the window level and window width during window conversion. FIG. 3 shows an overview of a window conversion-setting screen of the present embodiment. FIG. 2(A) shows the pixel intensity of 0-1,023 stages in image data in the transverse axis and a gray-scale of 256 stages that can be displayed in the longitudinal axis. Moreover, the line graph indicated with a bold line in FIG. 2(A) shows the correspondence relationship between the pixel intensity and the gray-scale.

The operator may perform a manipulation to change the shape of the graph showing the correspondence relationship between the pixel intensity and the gray-scale as shown in this graph in the window conversion-setting screen as shown in FIG. 3 via the manipulation part 201. With this manipulation, the display (e.g., brightness value) of the two-dimensional image can be adjusted. Namely, when the operator performs a manipulation to start adjusting the two-dimensional image via the manipulation part 201, the two-dimensional display-processing part 130 reads a screen format of the window conversion-setting screen as shown in FIG. 3 from a storing part that is not shown. Furthermore, the two-dimensional display-processing part 130 assigns image data that is to be window-converted to a two-dimensional image display region 310 in the screen format of the window conversion-setting screen as shown in FIG. 3 and sends it to the display part 202.

When the display part 202 receives the window conversion-setting screen and the image is displayed, the window conversion manipulation is possible. The operator may perform the window conversion manipulation on the window conversion-setting screen using a pointing device (such as a mouse), for example, as the manipulation part 201. The operator may refer to an image or a window display graph 320 displayed in the two-dimensional image display region 310 and execute the window conversion manipulation through a window conversion-setting region 330 via the manipulation part 201.

To a set S1 of a WL adjustment bar 331 and a WW adjustment bar 332, or a set S2 of a WW minimum value adjustment bar 333 and a WW maximum value adjustment bar 334 in the window conversion-setting region 330, the pixel intensities of the two-dimensional image have been assigned in stages. For example, to the WL adjustment bar 331, the WW adjustment bar 332, the WW minimum value adjustment bar 333, and the WW maximum value adjustment bar 334, pixel intensities from 0 to 1,023 stages, for example, have been assigned in the upward direction in FIG. 3.

Moreover, the position of “” assigned to each adjustment bar in FIG. 3 represents the pixel intensity of each adjustment bar.

For example, in the set S1, when the operator first attempts to set the pixel intensity corresponding to the median value of the gray-scale in the two-dimensional image, the operator adjusts the WL adjustment bar 331 in the window conversion-setting region 330 in the window conversion-setting screen. When the manipulation to adjust the median value of the gray-scale is done by the operator with the WL adjustment bar 331, the window conversion part 132 causes the pixel intensity corresponding to the adjusted window level to be reflected in the window display graph 320. Furthermore, the window conversion part 132 changes the two-dimensional image data so that a portion having a pixel intensity corresponding to the window level in the two-dimensional image is displayed in the median gray-scale.

Next, when the manipulation to adjust the window width is done by the operator with the WW adjustment bar 332, the window conversion part 132 causes the adjusted window width to be reflected in the window display graph 320. Furthermore, the window conversion part 132 changes the two-dimensional image data so that a portion of the two-dimensional image data below the pixel intensity set with the WW minimum value adjustment bar 333 is represented in black, for example. Similarly, the window conversion part 132 changes the two-dimensional image data so that a portion of the two-dimensional image data above the pixel intensity set with the WW maximum value adjustment bar 334 is represented in white, for example.

When the operator attempts to set the minimum and maximum values of the window width in the two-dimensional image with the set S2, the operator adjusts the WW minimum value adjustment bar 333 and the WW maximum value adjustment bar 334 in the window conversion-setting screen, respectively. When the manipulation to adjust the minimum and maximum values of the window width is done by the operator with the WW minimum value adjustment bar 333 and the WW maximum value adjustment bar 334, the window conversion part 132 causes the adjusted window width to be reflected in the window display graph 320. Furthermore, the window conversion part 132 changes the two-dimensional image data so that a portion of the two-dimensional image data below the pixel intensity set with the WW minimum value adjustment bar 333 is represented in black, for example.

Similarly, the window conversion part 132 changes the two-dimensional image data so that a portion of the two-dimensional image data above the pixel intensity set with the WW maximum value adjustment bar 334 is represented in white, for example.

In addition, in FIG. 2(A), which exemplifies the aforementioned window conversion, a pixel intensity that will be “127” in gray-scale as the window level has been set to “512”. Moreover, “340” has been set as the lower limit of the window width M and “640” as the upper limit. This range of 340≦M≦640 is the window width. With the window conversion part 132, gray-scale display is done in stages proportional to the pixel intensity within this range.

For the MRI device, window conversion processing is performed prior to volume rendering processing, for example.

Moreover, subjects of window conversion are T1-weighted images, T2-weighted images, and T2*-weighted images, for example.

In addition, for the ultrasound image-acquiring device, no window conversion is performed but gain adjustment and STC (sensitivity time control) adjustment are performed. However, by conceptually replacing the window width and window level adjustment in the window conversion-setting screen in the window conversion part 132 of the present embodiment 132 with gain adjustment and STC adjustment, the present embodiment can be applied to an ultrasound image-acquiring device. Hereafter, when applying this medical image-processing device to an ultrasound image-acquiring device, descriptions of the “window width” and “window level” will be replaced by “gain” and “STC”. Likewise, when applying this medical image-processing device to an ultrasound image-acquiring device, descriptions of “window conversion” will be replaced by “gain adjustment and/or STC adjustment”. In addition, the STC adjustment described above is synonymous with TGC (time gain compensation) adjustment.

Further, the above-mentioned window conversion part 132 is intended to apply the window conversion to “data stored in the image-storing part 121” and “data generated by two-dimensional image-generating part 131”. However, if the medical image processing device is applied to an ultrasonic image acquiring device, gain adjustment and STC adjustment are conducted at the timing of imaging.

Therefore, if the medical image processing device is applied to an ultrasonic image acquiring device, instead of the window conversion part 132, the imaging control part 110 and the image-acquiring part 112 (comprising an ultrasonic probe) conduct gain adjustment and STC adjustment. For example, based on manipulation by the manipulation part 201, the imaging control part 110, etc applies gain adjustment and STC adjustment to the signal in connection with received ultrasound.

The ultrasonic image acquiring device displays the ultrasonic image by gray-scale based on the gain adjustment value and the STC adjustment value.

When it is indicated that the window conversion process is completed with the manipulation by the operator via the manipulation part 201, the two-dimensional display-processing part 130 matches the parameters of the set window width and window level stored in the storing part (not shown) to the two-dimensional image subjected to the settings and causes them to be stored. Storing parameters of window width and window level comprises storing a figure of window curve by the window conversion as shown by the window display curve 320.

In addition, the window conversion processing described above has been described as processing of two-dimensional images, but window conversion processing of three-dimensional images is also similar. Namely, the operator, etc. makes adjustments in the window conversion-setting screen and the window conversion part 132 thereby performs window conversion with respect to three-dimensional images.

Further, the manipulation for the window conversion may comprise manipulations to change the figure of the window curve as shown by the window display curve 320 directly on the screen.

### (Process of Three-Dimensional Display Processing)

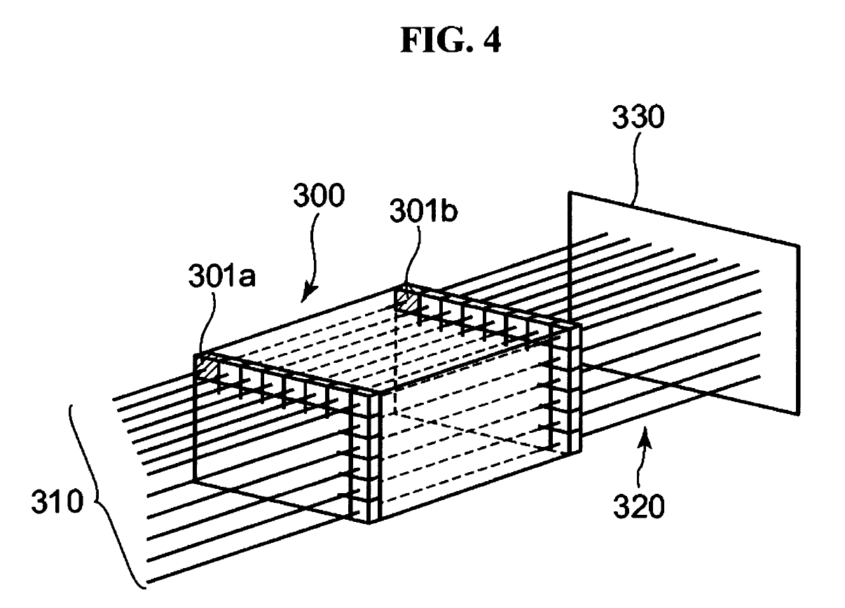

Next, image-processing and the setting of display conditions (window conversion) related to a three-dimensional display are described with reference to FIGS. 2(B), (C) and 4. FIGS. 2(B) and (C) are schematic diagrams showing an example of the relationship between signal intensity and opacity during volume rendering of a three-dimensional image. FIG. 4 is a schematic diagram showing an example of the viewpoint, line of vision, and projection plane during volume rendering.

The process of three-dimensional display processing will now be described with volume rendering as an example. When an instruction to display a three-dimensional image is provided by the operator via the manipulation part 201, a three-dimensional display-processing part 140 reads a screen format of a three-dimensional display-setting screen as shown in FIG. 4 from a storing part that is not shown. In addition, the three-dimensional display-processing part 140 reads volume data from the image-storing part 121. Furthermore, the three-dimensional display-processing part 140 generates the three-dimensional display-setting screen by assigning the volume data to the screen format of the three-dimensional display-setting screen. Furthermore, the three-dimensional display-processing part 140 sends data of the three-dimensional display-setting screen to the display part 202 and causes the display part 202 to display it.

The operator may perform various settings in volume rendering on the displayed three-dimensional display-setting screen via the manipulation part 201 (see FIG. 4). Various settings include setting of a viewpoint 310 with respect to volume data 300, a line of vision 320, a light source, or shading, for example. At a viewpoint setting part 141, so-called ray casting is performed with respect to an object (300) seen from the set viewpoint 310 based on information of the set light source, shading, and opacity, and pixel intensity for each pixel on a projection plane 330 is defined. When the pixel intensity is defined, the three-dimensional display-processing part 140 projects a three-dimensional image onto the projection plane 330. The projection plane 330 is a two-dimensional plane virtualized on the opposite side across the volume data 300 with respect to the set viewpoint 310.

In this volume rendering, in addition to setting the viewpoint, etc., setting of an opacity curve is also done. With the medical image-processing device of the present embodiment, an opacity curve-setting part 142 in the three-dimensional display-processing part 140 executes setting of the opacity curve and ray casting as follows using a set value for window conversion that has been set in advance.

First, the opacity curve-setting part 142 reads the parameters of the window width and window level set by the window conversion part 132 in the two-dimensional display-processing part 130 from the storing part (not shown). In addition, when the opacity curve-setting part 142 reads these parameters in this way, it refers to attached information, etc. attached to the image data. Furthermore, the opacity curve-setting part 142 set the correspondence relationship between the pixel intensity for each voxel 301 in the volume data and the opacity of the three-dimensional image display based on the window width and window level that have been read. In addition, this pixel intensity for each voxel 301 is hereinafter described as “voxel value”. Here, the opacity shown in FIG. 2(B) has been set in stages within the range from 0 to 1.0, with 0 defined as being completely transparent and 1.0 defined as being completely opaque. Moreover, setting by the opacity curve-setting part 142 is performed by setting each voxel value with respect to each stage of opacity in this range. In addition, the opacity curve-setting part 142 of the present embodiment is an example of the “opacity-setting part” according to the present invention. Further, the ultrasonic image acquiring device may conduct at least one of gain adjustment and STC adjustment, and the opacity curve setting part 142 acquires at least one of gain adjustment value and STC adjustment value.

Namely, the opacity curve-setting part 142 searches for each portion having the same voxel value as the pixel intensity corresponding to the window level (e.g., voxel 301). The opacity curve-setting part 142 assigns the median value of the aforementioned opacity (e.g., 0.5 opacity) to each portion having this voxel value corresponding to the window level. Furthermore, the opacity curve-setting part 142 sets the OWL with this assignment (see FIG. 2(B)). With this setting of the OWL, a voxel (such as 301) to which the median value of opacity is assigned in the three-dimensional image is defined.

Moreover, the opacity curve-setting part 142 searches for each portion having voxel value below the minimal value of the window width (e.g., voxel 301) as shown in FIG. 2(B). Furthermore, the opacity curve-setting part 142 assigns 0 for the aforementioned opacity to each portion having this voxel value below the minimal value. This defines a voxel to be displayed transparently in the three-dimensional image.

Likewise, the opacity curve-setting part 142 assigns 1.0 for the aforementioned opacity to each portion having voxel value above the maximum value of the window width. This defines a voxel to be displayed opaquely in the three-dimensional image. Moreover, an opacity proportional to voxel value is assigned to each portion having voxel value within the range of the window width. In this way, the OWW with respect to the three-dimensional image is set.

Furthermore, the opacity curve is set based on the OWW and OWL set by the opacity curve-setting part 142 (see FIG. 2(B)).

In addition, in FIG. 2(B), which exemplifies the aforementioned window conversion, the window level “512” in FIG. 2(A) has been utilized as the OWL as it is and the gray-scale in the window conversion and the opacity scale have been matched with each other.

Namely, the pixel intensity of the image data corresponding to the OWL is “512”, which is the same as the window level, and the opacity of “0.5” corresponding to the gray-scale of “127” in the window conversion has been set. Moreover, the window width from 340 to 640 in the window conversion has been utilized as the OWW as it is. With the opacity curve-setting part 142, within this OWW range, opacity display is done in stages proportional to the voxel value of each portion within the volume data.

That is, the opacity curve-setting part 142 of the present embodiment matches the parameters of window conversion to the opacity scale in the opacity curve while setting the opacity curve by replacing the shape of the graph of the window conversion shown in FIG. 2(A) with the shape of the opacity curve as shown in FIG. 2(B) (window curve). With this setting of the opacity curve, opacity display setting for each voxel is executed.

Furthermore, with the medical image-processing device of the present embodiment, it is also possible to change the shape of the graph (opacity curve) showing the correspondence relationship between the voxel value and opacity as shown in FIG. 2(B) and to make fine adjustments of the display of opacity in the three-dimensional image.

Namely, when the operator performs a manipulation to start adjusting the three-dimensional image via the manipulation part 201, the three-dimensional display-processing part 140 reads a screen format of an opacity curve change-setting screen (not shown) from a storing part that is not shown and causes the display 202 to display it.

Furthermore, the operator may perform the manipulation to change the opacity curve on the opacity curve conversion-setting screen via the manipulation part 201 (e.g., a pointing device, such as a mouse) in a similar manner to the window conversion described above.

In response to the change manipulation, the three-dimensional display-processing part 140 changes the opacity curve. In addition, the manipulation for the window conversion and for changing the opacity curve, it may be a manipulation executed by dragging the display of a threshold (such as window width or the minimum and maximum OWW values) on the graph as shown in FIGS. 2(A) and (B) using a pointing device, etc.

The imaging control part 110, the reconstruction part 120, the volume data-generating part 122, the two-dimensional display-processing part 130, and the three-dimensional display-processing part 140 in the configuration above are each composed of a memory that stores a program in which the content of the aforementioned operation has been described and a CPU that executes that program.

In addition, as shown in FIG. 2(C), the opacity curve-setting part 142 in the three-dimensional display-processing part 140 sets only the OWL to be the same as the window level, and the width of the OWW with respect to this may be configured to increase and decrease with respect to the window width. Namely, it may be configured to multiply the maximum and minimum values of the parameters of the set window width by a preset arbitrary coefficient and change the width of the OWW.

In addition, in FIG. 2(C), which exemplifies the aforementioned opacity setting, the window level “512” in FIG. 2(A) has been utilized as the OWL as it is and the gray-scale in the window conversion and the opacity scale have been matched with each other. Namely, the OWL is “512”, which is the same as the window level, and the opacity of “0.5” corresponding to the gray-scale of “127” has been set. In contrast. in FIG. 2(C), the upper and lower limits of the window width in the window conversion and the product of voxel value in this range and the coefficient “0.5” have been utilized as the OWW for setting the opacity curve. Therefore, in cases of such a configuration, compared to the case of FIG. 2(B) that utilizes the window width as it is, the range of the OWW has been set to be narrow with respect to the window width from 340 to 640. In addition, in this configuration, with the opacity curve-setting part 142, within this narrow range, opacity display is done in stages proportional to the voxel value of each portion within the volume data.

### [Actions and Effects]

For the medical image-processing device of the present embodiment described above, the operator sets the parameters of the window level and window width for displaying a two-dimensional image. The opacity curve-setting part 142 is configured to utilize these set parameters for setting the display conditions for a three-dimensional image (i.e., volume rendering). Specifically, the opacity curve-setting part 142 is configured to utilize these parameters for setting the opacity curve.

Therefore, for the medical image-processing device of the present embodiment, the operator only performs window conversion with respect to a two-dimensional image, thereby making it easy to set the opacity with respect to a three-dimensional image. Alternatively, the operator only performs window conversion with respect to a two-dimensional image and setting of the opacity can thereby be omitted.

### MRI Device

Moreover, for the MRI device, the pixel intensities vary greatly due to the types of pulse sequences, the TE (echo time) for each of the types, differences in parameters such as TR (repetition time), and differences in the settings of the transceiver coil for patients in addition to the tissues and conditions within the subject. In other words, for the MRI device, it is difficult to set preset parameters for the window conversion because there are many factors that define the pixel intensities. Further, it is also difficult to set preset parameter for opacity curve. Moreover, even if preset parameters are provided for setting the window conversion or opacity curve, in many cases, no image acceptable for viewing is obtained with the window conversion and opacity curve set according to the preset parameters alone.

For the above reasons, the image viewer has been required to make great efforts for setting the window conversion and opacity curve.

Moreover, appropriately setting the window conversion with the MRI device depends greatly on the experience of each individual viewer, which makes the setting task difficult.

Further, for an MRI device, in cases where a three-dimensional image is imaged by multi-slicing, it is common for window conversion manipulation to be performed with respect to two-dimensional T1-weighted images, T2-weighted images, and T2*-weighted images.

Moreover, with an MRI device, window conversion is sometimes done when imaging the three-dimensional image. If the medical image-processing device of the present embodiment is applied as an MRI device, the parameters related to window conversion set therein are utilized for setting the opacity curve during volume rendering. The inventor of the present invention has confirmed that this setting of the opacity curve was effective as presetting during volume rendering. As a result, setting of the opacity curve, which has conventionally been very difficult, will be simple. Alternatively, it will be possible to omit setting of the opacity curve. Therefore, the MRI device applying the medical image-processing device of the present embodiment can reduce the burden on the image viewer and also allow the efficiency of radiogram interpretation and diagnostic imaging to be improved.

### Ultrasound Image-Acquiring Device

For the ultrasound image-acquiring device, the pixel intensities vary greatly due to adjustments of the sound pressure level, receive gain, and STC (sensitivity time control) for gain correction with respect to only a portion in the depth direction in a multistep and an independent manner, etc. In other words, for the ultrasound image-acquiring device, it is difficult to set preset parameters for the window conversion and opacity curve. For the above reasons, the image viewer has been required to make great efforts for setting the window conversion and opacity curve. Moreover, appropriately setting the window conversion and opacity curve with the ultrasound image-acquiring device depends greatly on the experience of each individual viewer, which makes the setting task difficult.

Further, with an ultrasound image-acquiring device, if three-dimensional imaging is performed using a one-dimensional array of ultrasound transducers, gain adjustment and STC adjustment are performed by the operator. When performing this gain adjustment and STC adjustment, the operator first makes an adjustment while observing a two-dimensional image displayed in real time with a beam plane of the one-dimensional array of ultrasound transducers being fixed (without sways) so that it will be displayed properly. Here, if the medical image-processing device of the present embodiment is applied to an ultrasound image-acquiring device, the parameters (gain adjustment value and STC adjustment value) that have been set in gain adjustment and STC adjustment are once stored. Subsequently, for three-dimensional imaging, fluctuation of the ultrasound beam plane is started. With this start of fluctuation, a volume rendering display image is displayed per fluctuation in the unilateral direction. At this time, as the ultrasound image-acquiring device initially displays a three-dimensional image, the stored parameters are utilized for setting the opacity curve and volume rendering is done.

In addition, in cases of a two-dimensional array of ultrasound transducers, the ultrasound image-acquiring device forms a plane beam surface only in a cross-sectional position that will be a central cross-section, and performs gain adjustment and STC adjustment with respect to that cross-section. In addition, in this case, the ultrasound image-acquiring device applying the configuration of the present embodiment once stores the parameters set in the gain adjustment and STC adjustment. Subsequently, the ultrasound image-acquiring device updates images in real time by switching to a three-dimensional scanning mode, such as block sending and receiving, while displaying a three-dimensional image. At this time, the stored parameters are utilized for setting the opacity curve and volume rendering is done by the ultrasound image-acquiring device.

In addition, in cases where the medical image-processing device of the present embodiment is an ultrasound image-acquiring device, setting of the opacity curve, which has been very difficult conventionally, can be made simple or possible to omit. As a result, the ultrasound image-acquiring device can reduce the burden on the image viewer and also allow the efficiency of radiogram interpretation and diagnostic imaging to be improved.

### [Operation]

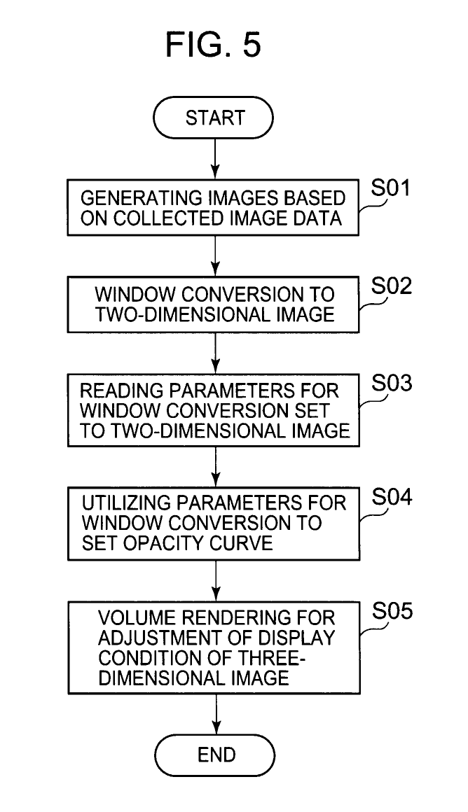

Operation of a medical image management device of the present embodiment as described above will now be described. FIG. 5 is a flow chart representing a series of operations of the medical image-processing device for explaining the tasks through which a user, such as an operator, performs display processing of a three-dimensional image using the medical image-processing device according to the present embodiment. Based on this FIG. 5, an example of an operation in a case where the medical image-processing device of the present embodiment is applied to an MRI device is described.

### [Step 1]

First, capturing conditions, such as pulse sequence, are set by the user (such as an operator), and an instruction to start imaging is executed via the manipulation part 201. Upon this instruction to start imaging, for example, a T2-weighted image with multiple slices is generated by the image-acquiring part 112 in the MRI device. This T2-weighted image is once stored in the image-storing part 121.

### [Step 2]

Next, based on the generated image data, an instruction to view the T2-weighted two-dimensional image is executed by the user via the manipulation part 201. Upon this instruction to view, the two-dimensional display-processing part 130 in the MRI device reads a screen format of a window conversion-setting screen (see FIG. 3) and the image data to be viewed and generates a window conversion-setting screen. Furthermore, the two-dimensional display-processing part 130 causes the display part 202 to display the generated window conversion screen. Furthermore, when the window conversion manipulation is done on the window conversion-setting screen by the user, the window conversion part 132 changes the gray-scale display according to the pixel intensity of the two-dimensional image data in response to the window conversion manipulation. Additionally, the window conversion part 132 causes the window display graph 320 to change the changed window level and window width. The parameters set here are stored in a storing part (not shown) along with attached information of the subject (such as patient ID, captured date, and information related to capturing conditions).

### [Step 3]

After the T2-weighted two-dimensional image is viewed by the user, if it is instructed by the user to display a T1-weighted image, for example, a T1-weighted image is generated through a similar process up to step 2. Furthermore, in this case, window conversion, etc. of the T1-weighted two-dimensional image is done by the window conversion part 132. Subsequently, if it is instructed by the user to display a T2-weighted three-dimensional image, for example, the parameters of window conversion set in step 2 are read based on the attached information of the subject stored in step 2.

### [Step 4]

The three-dimensional display-processing part 140 causes the display part 202 to display a T2-weighted three-dimensional image according to the manipulation. Furthermore, the three-dimensional display-processing part 140, in performing volume rendering, performs setting of the opacity curve with the parameters of window conversion that have been read. Namely, the opacity curve-setting part 142 matches the gray-scale corresponding to the window level and window width with the opacity scale in the opacity curve while assigning the parameters of the window level and window width to the setting of the opacity curve as they are. In this way, setting of the opacity curve is done.

### [Step 5]

Furthermore, the three-dimensional display-processing part 140 performs volume rendering based on the set opacity curve, the viewpoint 310, etc. (see FIG. 4) set by the user. When the three-dimensional display-processing part 140 performs volume rendering, volume data is projected onto the projection plane 330 and display conditions are adjusted in the T2-weighted three-dimensional image designated by the user.

### Second Embodiment

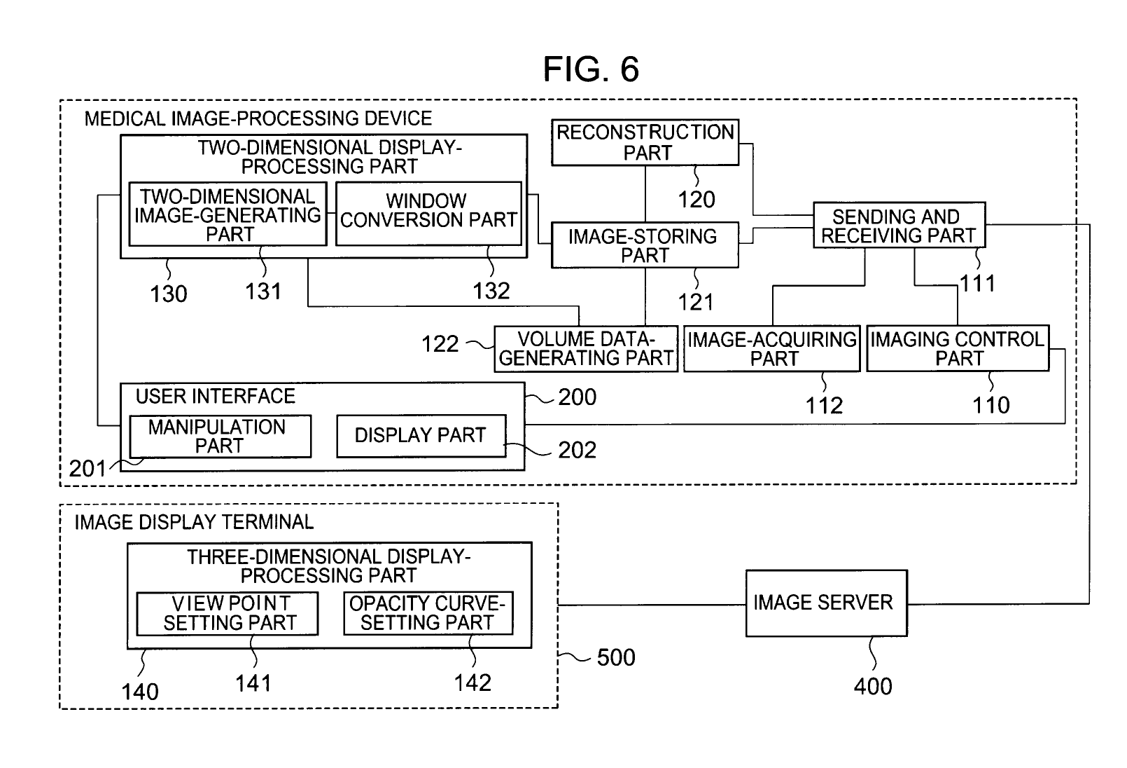

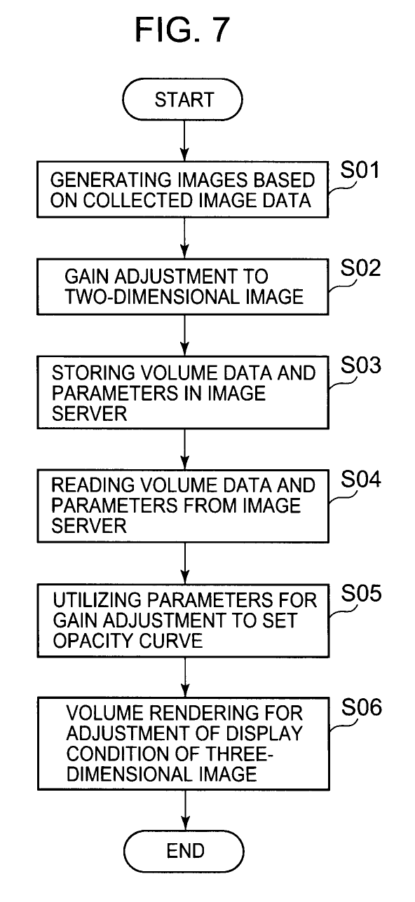

Next, the medical image-processing system according to the second embodiment of the present invention is described with reference to FIGS. 6 and 7. FIG. 6 is a block diagram showing the schematic conformation of a medical image-processing system according to the second embodiment of the present invention.

In the medical image-processing system according to the second embodiment, image data is generated by the image-acquiring part 112 in the medical image-processing device. The generated image data is sent to an image server 400 by the sending and receiving part 111 in the medical image-processing device and stored in the image server 400. Moreover, the image data stored in the image server 400 may be read from the image server and displayed on an image display terminal 500.

An example of an operation in a case where the medical image-processing device in the medical image-processing system of the present embodiment is applied to an ultrasound image-acquiring image is described as follows with reference to FIG. 7. FIG. 7 is a flow chart representing a series of operations of the medical image-processing device for explaining the tasks through which the user, such as an operator, performs display processing of a three-dimensional image using the medical image-processing device of the present embodiment.

In addition, in this explanation of the operation, the window conversion part 132 is substituted with a gain/STC conversion part for convenience.

### [Step 1]

First, capturing conditions for the imaging method, such as the Doppler mode or B-mode (brightness mode), are set by the operator, and furthermore, an instruction to start imaging is executed via the manipulation part 201. Upon receiving the instruction to start imaging, the collection of information within the subject's body is started by the image-acquiring part 112 in the ultrasound image-acquiring device.

### [Step 2]

In cases where three-dimensional imaging is performed using a one-dimensional array of ultrasound transducers, the operator performs gain adjustment and STC adjustment while observing a two-dimensional image displayed in real time with a beam plane being fixed. Namely, when gain adjustment value and STC adjustment value are set on the generated data by the operator via the manipulation part 201, the two-dimensional display-processing part 130 in the ultrasound image-acquiring device executes gain adjustment for the two-dimensional image data in response to the gain adjustment manipulation. Moreover, the two-dimensional display-processing part 130 executes STC adjustment in response to STC adjustment manipulation. The parameters set here are stored in a storing part (not shown) along with attached information of the subject (such as patient ID, captured date, and information related to capturing conditions).

### [Step 3]

When three-dimensional imaging is completed, the ultrasound image-acquiring device as the medical image-processing device sends gain adjustment value, STC adjustment value and attached information that have been stored and the generated volume data to the image server 400 via the sending and receiving part 111. In the image server 400, the volume data and parameters are matched with the attached information and stored.

### [Step 4]

The user performs a manipulation for an instruction to read a three-dimensional image along with attached information at the image display terminal 500 in order to view the three-dimensional image generated at the ultrasound image-acquiring device. The image display terminal 500 sends the read instruction to the image server 400 in response to the manipulation. The image server 400 sends the volume data and parameters that have been stored based on the attached information related to the read instruction to the image display terminal 500. Moreover, the three-dimensional display-processing part 140 in the image display terminal 500 causes a three-dimensional image to be displayed based on the volume data received in response to the manipulation.

### [Step 5]

Furthermore, the three-dimensional display-processing part 140, when performing volume rendering, performs setting of the opacity curve. The three-dimensional display-processing part 140 performs this setting of the opacity curve based on gain adjustment value and STC adjustment value that have been read. Namely, the opacity curve-setting part 142 matches gray-scale based on gain adjustment value and STC adjustment value with the opacity scale in the opacity curve while assigning the parameters of gain adjustment and the parameters of STC adjustment to the setting of the opacity curve as they are. In this way, setting of the opacity curve is done.

### [Step 6]

Furthermore, the three-dimensional display-processing part 140 performs volume rendering based on the set opacity curve, the viewpoint 310, etc. (see FIG. 4) set by the user. When the three-dimensional display-processing part 140 performs volume rendering, volume data is projected onto the projection plane 330 and display conditions are adjusted in the three-dimensional image designated by the operator.

### [Actions and Effects]

As described above, with the medical image-processing device according to the second embodiment, setting of the opacity curve, which has been very difficult conventionally, can be made simple or possible to omit. As a result, with the medical image-processing device of the present embodiment, the burden on the image viewer can be reduced, and it is possible to improve the efficiency of radiogram interpretation and diagnostic imaging.

Moreover, the medical image-processing device of the second embodiment, when causing the volume data to be stored in the image server 400, is configured to cause the volume data and the parameters of gain and STC adjustment or the parameters of window conversion to be stored along with the attached information. Therefore, even when changing display conditions in order to view a three-dimensional image at a later date in a display device configured externally to the medical image-processing device, it will be possible to simplify or omit setting of the opacity curve, which has been very difficult conventionally.

In addition, it is naturally possible to apply the medical image-processing device in the first and second embodiments described above to an X-ray CT device. Next, an embodiment in which the medical image-processing device described above is applied to an X-ray CT device while being modified into a configuration particularly compatible with an X-ray CT device will be described.

### Third Embodiment

Next, an X-ray CT device as the medical image-processing device according to the third embodiment of the present invention will be described. With the X-ray CT device for performing medical image processing according to the third embodiment, image data is generated by the image-acquiring part 112 in a similar manner to the volume data generation process described above. Moreover, this image data undergoes window conversion as two-dimensional display processing, but for this X-ray CT device, window conversion is done as follows.

For the X-ray CT device, before scanning by the image acquiring part 112, parameters of various conditions such as scanning condition, reconstruction condition, and display condition are set via the manipulation part 201 in order to acquire the X-ray CT images. For example, for the X-ray CT device of the present embodiment, before acquiring the X-ray CT images, parameters such as slicing positions, a scope of imaging, a tube voltage, and a tube current are set. In addition to these parameters, parameters for window conditions, i.e. presetting the window level value and the window width value for the window conversion are set in advance. For the X-ray CT device of this embodiment, the window level value and the window width value are used for setting the opacity curve of volume rendering process. The opacity curve is set by the following procedure.

For this X-ray CT device, presetting the parameters of the window level and window width has been set in advance. The presetting has been stored in a storing part that is not shown. The window conversion part 132 reads the presetting related to window conversion when performing window conversion of image data generated in the volume data generation process. The window conversion part 132 performs window conversion of the image data based on the presetting that have been read and performs gray-scale processing of the image data.

In addition, with the X-ray CT device of the present embodiment, it is possible for the operator to adjust the parameters for window conversion on the window conversion-setting screen described above. For example, with the X-ray CT device, after executing a gray-scale display of a two-dimensional image with the presetting for window conversion, it is possible for the operator to make fine adjustments of the window conversion on the window conversion-setting screen, etc. Further, since variation of CT value may be expected due to imaging condition, temperature, beam hardening correction and etc, gray-scale processing of image data is conducted based on adjustment value in order to adjust the presetting value.

In addition, in this embodiment, the parameters set in the window conversion process are matched with image data and stored as attached information. Moreover, the process of applying the parameters of window conversion matched with the image data and stored to the opacity curve is as described above.

In an X-ray CT image generated by the X-ray CT device, a CT value (HU) indicating the pixel intensity becomes almost constant depending on the subject's tissue. Therefore, in the X-ray CT image, the relationship between the intensity of the image signal in each portion of the image and the gray-scale/opacity is defined almost unambiguously. As a result, the X-ray CT device is highly compatible with the aforementioned configuration.

### [Actions and Effects]

As described above, with the medical image-processing system according to the third embodiment, setting of the opacity curve, which has been very difficult conventionally, can be made simple or possible to omit. As a result, with the medical image-processing device of the present embodiment, the burden on the image viewer can be reduced, and it is possible to improve the efficiency of radiogram interpretation and diagnostic imaging.

Furthermore, for the medical image-processing device according to the third embodiment, presetting for window conversion has been set in advance, making it possible to further reduce the burden on the image viewer. In addition, in the present embodiment, a case where the medical image-processing device is an X-ray CT device has been described, but it is also possible to have the medical image-processing device of the present embodiment be an MRI device or an ultrasound image-acquiring device.

### Fourth Embodiment

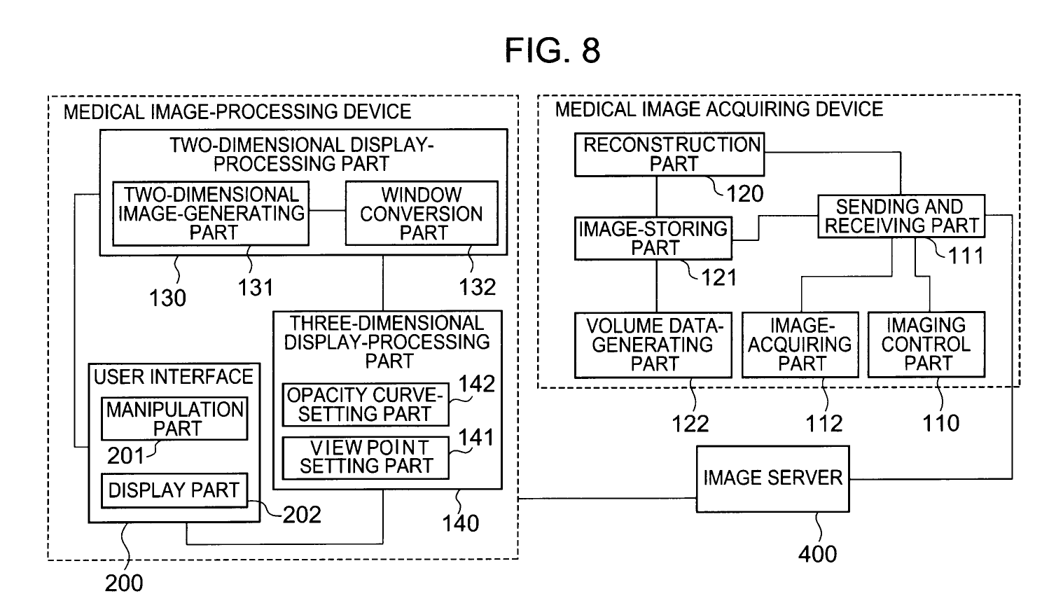

Next, the medical image-processing system according to the fourth embodiment of the present invention will be described with reference to FIG. 8. FIG. 8 is a block diagram showing the schematic conformation of the medical image-processing system according to the fourth embodiment of the present invention.

As shown in FIG. 8, the medical image-processing system according to the fourth embodiment is configured with the medical image-acquiring device, the medical image-processing device, and the image server 400. Herein, in the medical image-processing system of the present embodiment, the medical image-acquiring device is mainly for acquiring medical images and not necessarily for performing three-dimensional display processing, such as volume rendering, for viewing. Volume rendering is performed by the medical image-processing device that performs display processing of image data. The medical image-processing system of the present embodiment is described as follows based on FIG. 8.

As shown in FIG. 8, the medical image-acquiring device of the medical image-processing system receives settings for capturing conditions from the operator. The imaging control part 110 controls the image-acquiring part 112 to cause it image the subject and detects an image signal. Furthermore, the reconstruction part 120 in the medical image-acquiring device generates image data by performing reconstruction processing on the image signal. The generated image data is stored in the storing part 121. Furthermore, the medical image-acquiring device generates volume data with the volume data-generating part 122 based on the stored image data.

The medical image-acquiring device sends the generated volume data to the image server 400 with the sending and receiving part 111.

The image server 400 stores the received volume data. The image server 400 stores the volume data in a readable manner. In addition, the parameters of the window level, window width, and opacity prior to adjustment have been attached to the volume data.

The medical image-processing device can read the image data stored in the image server 400. When the volume data is read by the medical image-processing device, it undergoes three-dimensional display processing to make it viewable. The medical image-processing device performs window conversion related to gray-scale display with the two-dimensional display-processing part 130. This window conversion processing is as described above.

Moreover, the medical image-processing device performs volume rendering on volume data with the three-dimensional display-processing part 140. Setting of the opacity in this case utilizes the parameters set during window conversion. This aspect is also as described above. In addition, the medical image-processing device of the present embodiment is configured to perform window conversion with the medical image-processing device. However, the medical image-processing system of the present embodiment is not limited to this configuration. For example, it is also possible to employ the configuration to perform window conversion with the medical image-acquiring device. In this case, the parameters set during window conversion are attached to volume data as attached information, such as DICOM (Digital Imaging and Communication in Medicine). This attached information is stored in the image server 400 along with the volume data.

### [Actions and Effects]

As described above, with the medical image-processing system according to the fourth embodiment, setting of the opacity curve, which has been very difficult conventionally, can be made simple or possible to omit. As a result, with the medical image-processing device of the present embodiment, the burden on the image viewer can be reduced, and it is possible to improve the efficiency of radiogram interpretation and diagnostic imaging.

What is claimed is:

1. A medical image-processing device comprising:
information acquiring part configured to acquire a window level value and a window width value of medical image data of a subject;
opacity setting part configured to set a opacity curve of volume rendering based on the window level value and the window width value; and
volume rendering part configured to apply volume rendering process to the medical image data based on the opacity curve set by the opacity setting part.

2. The medical image-processing device according to claim 1, wherein:
the medical image data is MRI image data or X-ray CT image data, and
the information acquiring part is configured to acquire the window level value and the window width value of a window adjustment applied to the medical image data.

3. The medical image-processing device according to claim 1, wherein:
the opacity setting part is configured to utilize the window level value for median of the opacity of the opacity curve.

4. The medical image-processing device according to claim 2, wherein the opacity-setting part is configured to multiply a predetermined coefficient by the window width value in order to utilize the result as a width value of the opacity of the opacity curve.

5. The medical image-processing device according to claim 1, wherein the opacity-setting part is configured to utilize the window level value and the window width value of attached information added to the medical image data as a parameter for setting the opacity curve.

6. The medical image-processing device according to claim 2, wherein the opacity-setting part is configured to set the opacity curve based on the window level value and the window width value which is obtained when the window adjustment is applied to a MPR image or a tomographic image

7. The medical image-processing device according to claim 1, wherein:
the information acquiring part is configured to acquire the window level value and the window width value set in advance before acquiring the medical image data.

8. An ultrasonic image-acquiring device comprising
information acquiring part configured to acquire gain adjustment value and STC adjustment value when at least one of gain adjustment and STC adjustment of ultrasonic image collection of a subject is conducted;
opacity setting part configured to set a opacity curve based on the gain adjustment value or the STC adjustment value; and
volume rendering part configured to apply volume rendering process to the medical image data based on the opacity curve.

9. An ultrasonic image-acquiring device according to claim 8, wherein the opacity setting part is configured to utilize the gain adjustment value for median of the opacity of the opacity curve.

10. An ultrasonic image-acquiring device according to claim 8, wherein the opacity-setting part is configured to multiply a predetermined coefficient by the STC adjustment value in order to utilize the result as a width value of the opacity of the opacity curve.

11. An ultrasonic image-acquiring device according to claim 8, wherein the opacity-setting part is configured to utilize the gain adjustment value or the STC adjustment value of attached information added to the medical image data as a parameter for setting the opacity curve.

12. A medical image-processing method comprising:
acquiring a window level value and a window width value of medical image data of a subject;
setting a opacity curve of volume rendering based on the window level value and the window width value; and
applying volume rendering process to the medical image data based on the opacity curve.
